(19) United States
(12) Patent Application Publication
(10) Pub. No.: US 2018/0325435 A1
(43) Pub. Date: Nov. 15, 2018

(54) WIRELESS OXIMETER FOR CONTINUOUS USE

(71) Applicant: TÁCITO MISTRORIGO DE ALMEIDA, Sao Paulo (BR)

(72) Inventor: TÁCITO MISTRORIGO DE ALMEIDA, Sao Paulo (BR)

(21) Appl. No.: 15/774,683

(22) PCT Filed: Aug. 25, 2016
(86) PCT No.: PCT/BR2016/050210

§ 371 Date: May 9, 2018

(30) Foreign Application Priority Data
Nov. 11, 2015 (BR) BR1020150283725

(51) Int. Cl. A61B 5/1455; A61B 5/00; A61B 5/053

(52) U.S. Cl. CPC A61B 5/14552; A61B 5/0004; A61B 5/742; A61B 5/6843; A61B 2560/0456; A61B 5/0531; A61B 5/6824; A61B 2560/0214; A61B 5/7285

(57) ABSTRACT

The present invention provides a wireless pulse oximeter for continuous use comprising a first device (10), adapted to a part of the body of a patient and comprising physiological data sensoring means, communicating with a second remote device (12) by means of an RF connection (11), said first device powered by a rechargeable battery (31), being provided with battery charge saving means by automatically shutting off the power from the circuits when this device is not installed on the patient's body. The second remote device is an intelligent terminal containing a display as well as a specific application for processing the physiological data captured by the first device and received by way of said RF connection.

### FIELD OF THE INVENTION

The present invention refers to a wireless pulse oximeter (device that measures the saturation of oxygen and the heartbeat rate of a patient in a non-invasive manner), for continuous use, light and compact, easy to use by the average person, low cost, which collects information continuously and which automatically relays, by radio frequency, this information to a remote intelligent device (such as a smartphone or tablet). The information can be visualized on the screen of the intelligent device and can be relayed, also automatically, to Internet servers. Reports on the information collected can be generated on the intelligent device or on Internet servers.

### STATE OF THE ART

The pulse oximeters available on the market are basically of 3 types:

Non-portable—in the shape of a monitor, having a wireless sensor and are commonplace in hospitals.

Portable—having the display and the sensor in a single device.

Wearable—formed by two modules, one being the wired sensor installed on the finger, connected to a larger unit installed on the wrist. They enable continuous monitoring, but are very costly.

The first two alternatives above do not enable continuous monitoring or during sleep, in a comfortable and reliable manner. The third alternative enables continuous monitoring, but at a high cost.

Among the patent documents describing said devices, the following hold prominence.

U.S. Pat. No. 8,315,682—INTEGRATED PULSE OXIMETRY SENSOR, owned by KONINKL PHILIPS, describes a hollow tubular-shaped device, in which a finger is encased. Said device is endowed with two light emitting diodes that emit red and infrared light which, after traversing the finger, reflect on photodetectors which convert the radiations into electrical signals. After A/D conversion, these signals are forwarded to a processor integrated to the device which, by means of a specific software, determines the oxygenation content in the blood. This information is forwarded to a radio transmitter which relays it to a remote equipment for displaying and possible additional processing. The drawbacks of the subject matter of this patent include the lack of adaptability to the size of the finger, as individual anatomical variations are large.

U.S. Pat. No. 6,731,962—FINGER OXYMETER WITH REMOTE TELECOMMUNICATIONS CAPABILITIES AND SYSTEMS THEREFOR, filed by Smiths Medical describes a system in which the RF signal relayed by the finger oximeter is received by a remote device, such as, for example, the Vital Signs Monitor produced by the same owner. Besides displaying the relevant information, the remote device enables control of the working of the finger oximeter, by sending a signal that activates/deactivates the finger oximeter. The system described in this patent has a display on the device itself, which implies drawbacks, such as greater battery consumption, and larger size and weight of the device, which compromises comfort and bars the use thereof for continuous monitoring or during the night. Additionally, it does not have an internal accelerometer for detecting motion and the consequent improvement in the quality of the information that is generated.

Patent document US2010125188—MOTION CORRELATED PULSE OXIMETRY, filed by Nonin Medical Inc., describes a device comprising a first sensor, a motion sensor and a processor. The first sensor has an optical detector and an optical emitter. The optical detector generates a first output using the optical emitter. The first output corresponds to a physiological parameter of a user such as, for example, the saturation of oxygen in the blood. The motion sensor generates a motion output corresponding to a detected motion of the user. The motion sensor is configured for attachment to the user. The processor is coupled to the first sensor by a first link and coupled to the motion sensor by a second link. The device also comprises an RF communication connection. The invention described therein is separated into two parts interlinked by a wire, one part fastened to the patient's wrist and the other part being a conventional sensor fastened to the patient's finger. Besides the problems deriving from potential wear of the wire, this arrangement is very costly.

It is therefore noted that none of the alternatives above enable continuous monitoring, even during sleep, in a comfortable and reliable manner and at low cost.

### OBJECTIVES OF THE INVENTION

In view of the shortcomings and drawbacks noted in the devices known in the state of the art, it is a first objective of the invention to enable patients' variables, such as saturation of oxygen, heartbeat rate and motion, to be monitored continuously, even during sleep.

Another objective consists of providing greater ease of use and improved comfort for the patient.

Another objective consists of providing an oximeter that adapts to the various measurements of the patient's finger and which is not easily unfastened.

Another objective consists of enabling the data captured by the system to be made available in an application (app) of intelligent devices and on the Internet.

### GENERAL DESCRIPTION OF THE INVENTION

The pulse oximeter is an instrument to estimate the saturation of arterial oxygen of a patient's blood, in a non-invasive manner. As such, at least two light sources are used, having different wavelengths and a photo-sensor. Since the light absorption of oxygenated hemoglobin and deoxygenated hemoglobin is different in the two wavelengths, by using this difference it is possible to determine the saturation of oxygen in the blood.

The present invention uses the conventional method described above, but fills a gap existing on today's market, which is an oximeter that enables continuous monitoring of the saturation of arterial oxygen, that is, for several hours and even during sleep, being low cost, easy to use by untrained persons and high-precision.

The subject matter of the invention is a pulse oximeter composed of two main parts: the first consists of a wireless sensor, and the second of an application (app) which is installed in a remote equipment provided by an intelligent communications terminal, such as, for example, a smartphone or a tablet.

In order to enable continuous use, the sensor is comfortable and accordingly it is wireless, light and compact. Since it has internal faces angled to accompany the finger contact surfaces, the upper and inner outer faces are parallel, and so a tape or elastic can be used to fasten it, assuring that the oximeter does not come off the finger, even during continuous use or during sleep.

Eliminating redundant elements enables a compact and low-cost device to be produced. Such is the case of using the screen of the remote device for displaying the data, enabling the display on the sensor installed on the patient's finger to be eliminated. By eliminating the display on the sensor, the battery is reduced, which also lowers costs and the size of the sensor.

To facilitate use by untrained persons, the sensor is switched on and off automatically, relays the information to the remote device in a transparent and automatic manner, and has its battery recharged in a simple manner by optionally using a docking station similar to that of wireless telephones.

### DESCRIPTION OF THE DRAWINGS

The advantages and features of the invention will become more apparent from the description of preferred embodiments, provided as an example and not as a limitation, and from the drawings which refer to them, wherein:

FIG. 1 illustrates the block diagram of the system of the present invention.

FIG. 2 illustrates the block diagram of the sensor device installed on the finger.

### DETAILED DESCRIPTION OF PREFERRED EMBODIMENTS

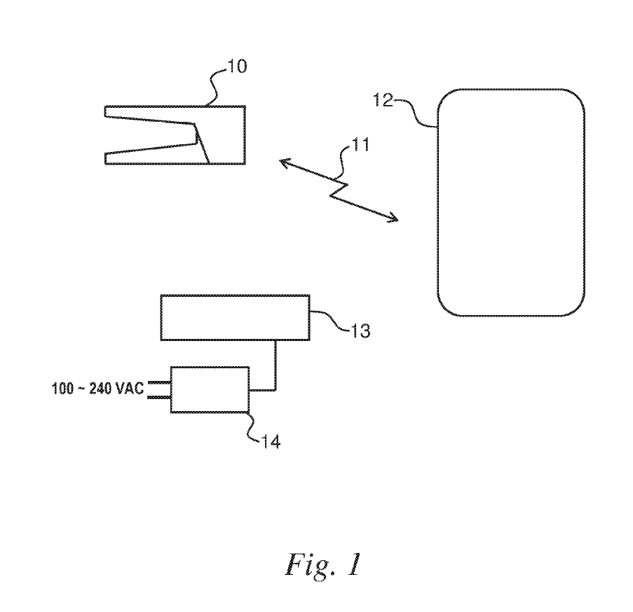

As it can be seen in FIG. 1, the sensor device (10) which is mounted on the finger of a patient's hand has a shape where the inner faces are angled in relation to each other, which enables the upper and lower outer faces to be parallel, allowing it to be fastened with the aid of tape or elastic. Said device collects and processes the data from the photo-sensors, calculating information such as the saturation of peripheral SpO2 oxygen, the heartbeat rate and motion of the sensor. This information is relayed continuously, via radio frequency connection (11) using, for example, technology known as Bluetooth Low Energy, to an intelligent device (12).

The intelligent device (12) has installed thereon an application dedicated to this functionality, having the following functions: reception of data sent by the sensor device, remote configuration of the sensor device, update of firmware of the sensor device, monitoring the communication link with the sensor device, monitoring the battery of the sensor device, presentation of data sent by the sensor device, report generation, monitoring of the intelligent device, relay of information to Internet servers and temporary data storage, among others.

The sensor device has a rechargeable battery with adequate duration. To recharge the battery, the sensor device should be removed from the patient's finger and placed on the docking station (13), which is connected to an AC/DC converter (14) powered by the conventional electricity grid. The user is able to know when the battery is charged by way of a LED indicator (30) on the sensor device (10) or by the application installed on the intelligent device.

When the patient wishes to use the sensor device, he/she just withdraws it from the docking station and places it on the finger. As soon as the presence of the finger is detected by the sensor device, it turns on automatically. When the sensor device is withdrawn from the finger, it automatically shuts down after a certain period, such as, for example, 10 seconds, as detailed ahead.

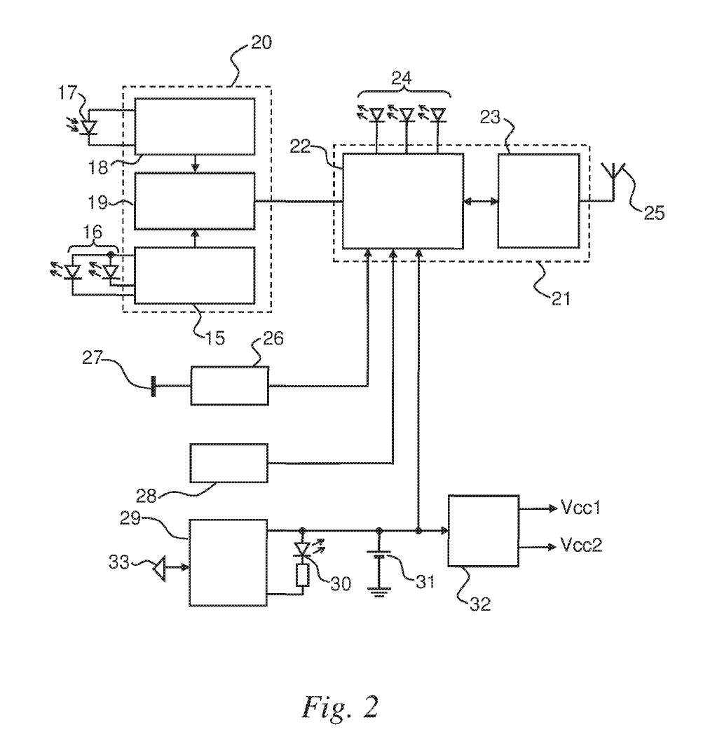

FIG. 2 presents the block diagram of the sensor device. The main components are the integrated circuit (20) “analog front end”, the integrated circuit (21) that contains a microcontroller (22) and a radio transceiver (23), the photodiode (17) associated to an amplifier and A/D converter (18), the double emitter (16) that contains a light emitting diode having a wavelength of 660 nm (red) and a light emitting diode having a wavelength of 940 nm (infrared), both activated by drivers commanded by a timer included in the block (15). The latter, as well as block 18, are connected to an interface controller block (19) which communicates with the microcontroller (22).

Besides these components, the sensor device is endowed with three LEDs (24) that indicate various error situations during use, an antenna (25) associated to the radiofrequency transceiver (23), an accelerometer (28) which detects motion of the sensor to enable improved quality of the information that is generated, a battery-charging integrated circuit (29), a connector (33) for adapting to the docking station, a lithium ion rechargeable battery (31) and an AC/DC converter set (32) which generates power for the various parts of the sensor.

The means that provide the automatic on/off when placing or withdrawing the patient's finger can respectively be provided by an ultralow-consumption capacitive sensor (26) and (27) that will indicate the presence of absence of the part of the patient's body which will be adjacent to the inner face of the sensor when it is installed. In this case, said capacitive sensor should operate continuously, or at intervals of a few seconds, maintaining the other blocks on stand-by, and they will only leave this low-consumption state when the capacitive sensor detects that the device has been installed on the finger.

In an alternative embodiment, a touch switch can be used, activated by pressing the inner face of the sensor against the skin of the patient when installing the device. This embodiment enables economy of the battery load, since all the circuits and elements of the sensor—including the capacitive sensor—will remain switched off and, therefore, will not consume power from the battery.

Although the present invention was described in connection with preferred embodiments, it should be understood that the invention is not meant to be limited to these particular embodiments. Therefore, for example, the sensor can be configured to adapt to other parts of the patient, such as the toes, earlobe etc., maintained within the conceptual limits of the invention. Accordingly, all the alternatives, modifications and potential equivalents within the spirit and scope of the invention are meant to be encompassed, as defined in the accompanying set of claims.

1. A WIRELESS PULSE OXYMETER FOR CONTINUOUS USE comprising a first device (10), adapted to a part of the body of a patient and comprising physiological data sensoring means (16, 17) and communication means (23, 25) powered by a rechargeable battery (31), and a second remote device (12) endowed with communication means with the first device wherein said communication means are provided by an RF connection (11), said second device consisting of an intelligent terminal comprising a display and a specific processing application for physiological data received by way of said RF connection, said first device comprising automatic battery charge saving means (31).

2. The WIRELESS PULSE OXYMETER FOR CONTINUOUS USE of claim 1, wherein said remote device (12) displays on a screen the information relating to the patient's physiological data and is selected from the group comprising a smartphone, a tablet and a computer.

3. The WIRELESS PULSE OXYMETER FOR CONTINUOUS USE of claim 1, wherein said automatic charge saving means (31) comprise a sensor (26, 27) activated by the presence of the part of a patient's body next to the inner face of said first device (10).

4. The WIRELESS PULSE OXYMETER FOR CONTINUOUS USE of claim 3, wherein said sensor is a capacitive sensor (26).

5. The WIRELESS PULSE OXYMETER FOR CONTINUOUS USE of claim 1, wherein said sensor is a switch (27) activated by pressing the inner face of said device against the skin of the patient.

6. The WIRELESS PULSE OXYMETER FOR CONTINUOUS USE of claim 1, wherein said first device (10) comprises battery recharging means (31), which comprise coupling means (33) to a docking station (13).

7. The WIRELESS PULSE OXYMETER FOR CONTINUOUS USE of claim 1, wherein the inner faces of the device (10) are angled in relation to each other accompanying the contour of the finger contact surfaces and enables the upper and lower outer faces of said first device (10) to be parallel.
